(19) United States
(12) Patent Application Publication
(10) Pub. No.: US 2004/0210115 A1
(43) Pub. Date: Oct. 21, 2004

(54) Disposable plastic laryngoscope with built in refractroy light source for the examination of the larynx

(76) Inventors: Thomas Tat Cheong Ma, Hong Kong (HK); William Y.W. Lee, Corona Del Mar, CA (US); Bryn Jarald Henderson, Orange, CA (US)

Correspondence Address: WILLIAM LEE, 341 EVENING CANYON, CORONA DEL MAR, CA, 92625, US

(21) Appl. No.: 10/417,618

(22) Filed: Apr. 18, 2003

(51) Int. Cl. A61B 1/267

(52) U.S. Cl. 600/199; 600/191

(57) ABSTRACT

This invention relates to the development of a plastic disposable laryngoscope with built in power source in any form and built in refractory light sources. This invention relates to the development of a plastic disposable laryngoscope (this can be constructed of wood, metal, rubber, plastic or any other disposable material) contains three major components: first component—plastic rotational light source cover that may be in various forms (e.g. press button) and on different locations of the plastic handle. Second component—plastic handle contains the power source in any form and the light bulb in different intensities. The third component is the plastic transparent blade, which connects to the base of the plastic handle. The plastic blade contains a refractory mirror at any angle that receives and refracts the light from any light source and delivers the light along the transparent plastic blade. The light intensity is double or more than the candlelight intensity from the regular non-disposable laryngoscope. This novel disposable plastic laryngoscope is practical, economical and gives the operator the best view of the larynx. For more detailed description of this invention please refer to the submitted drawings.

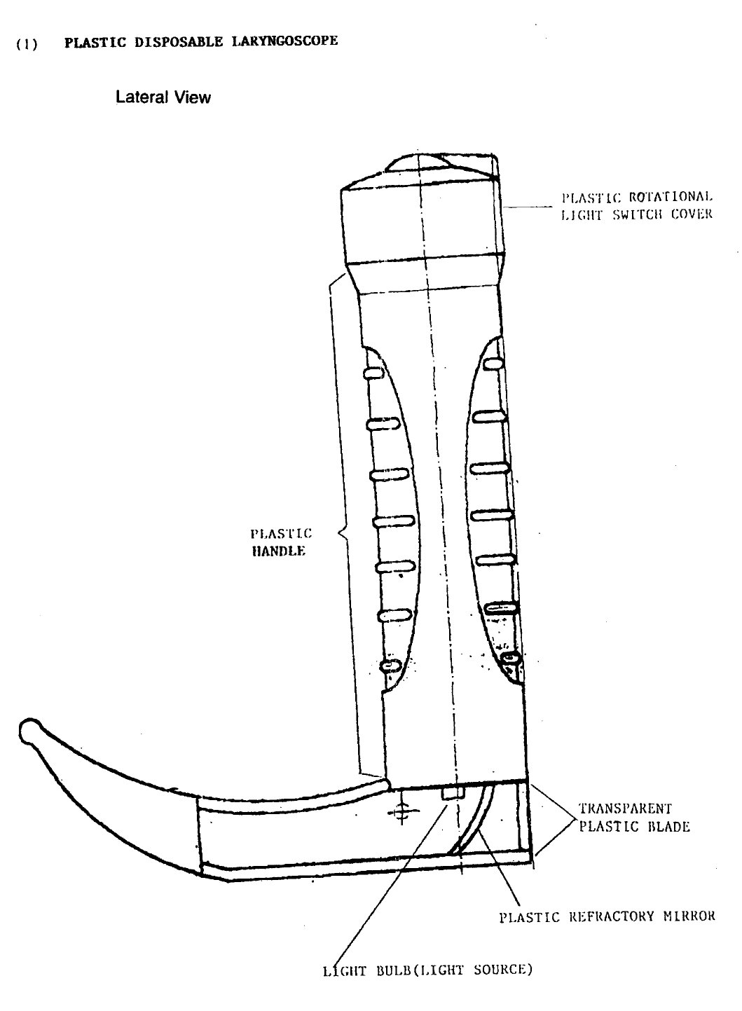

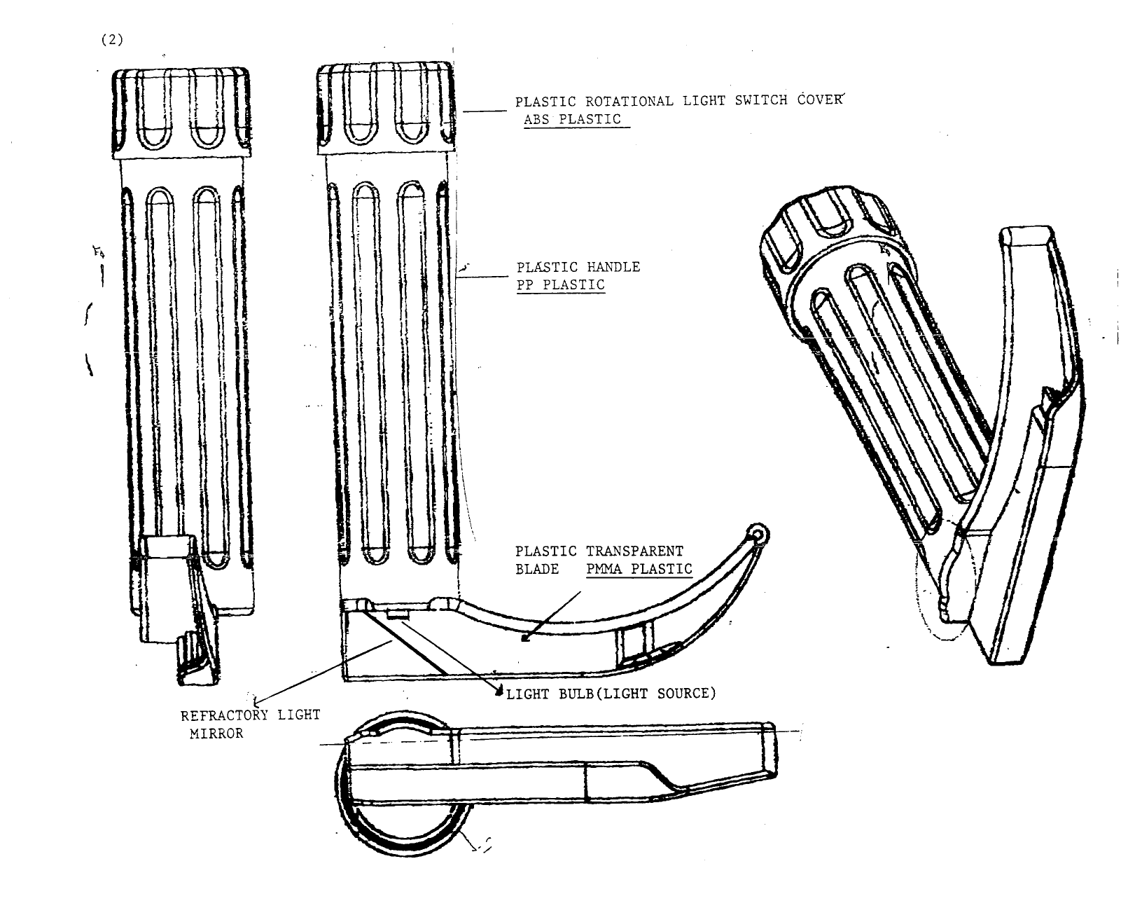

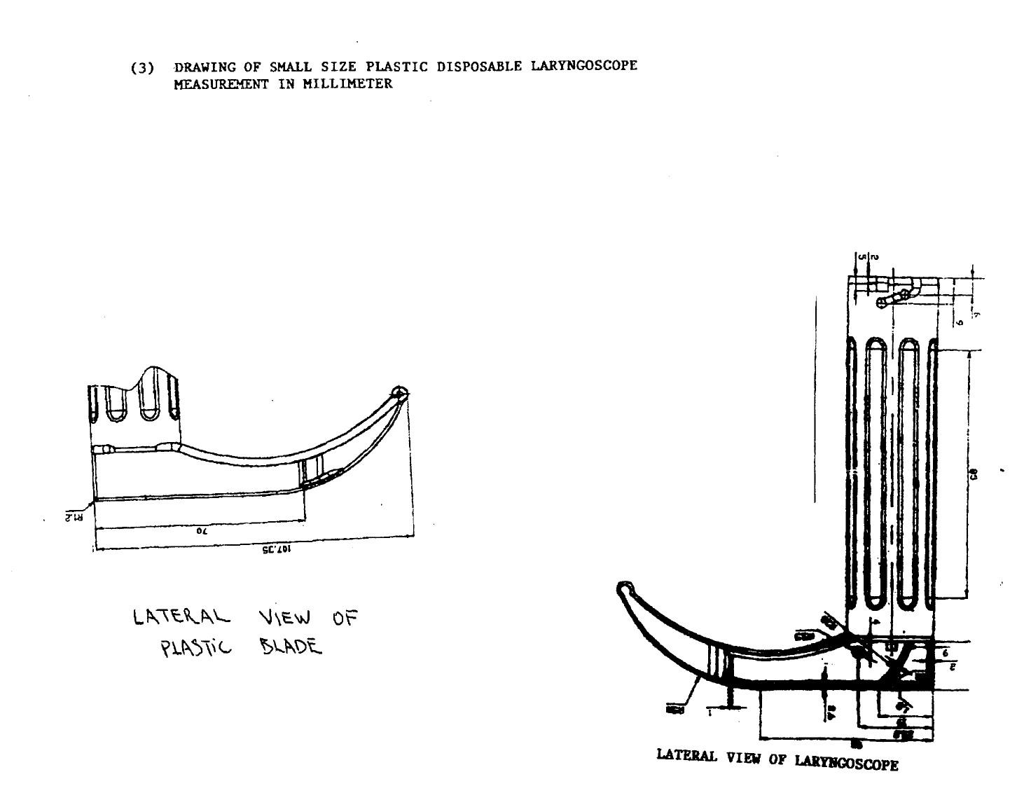

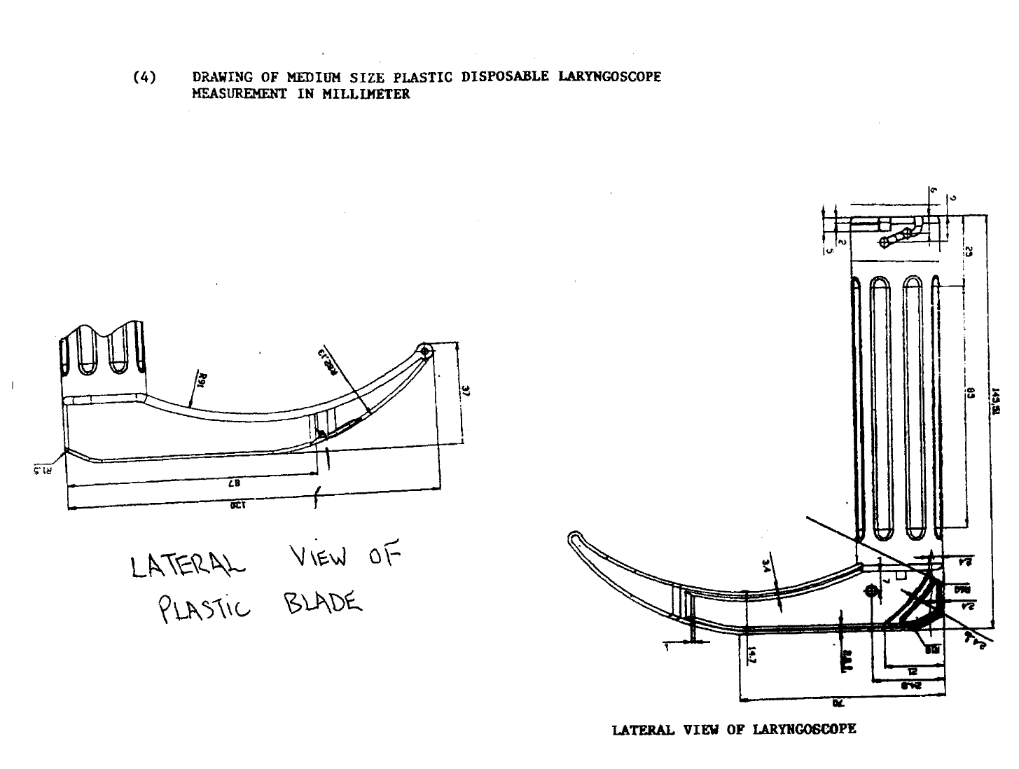

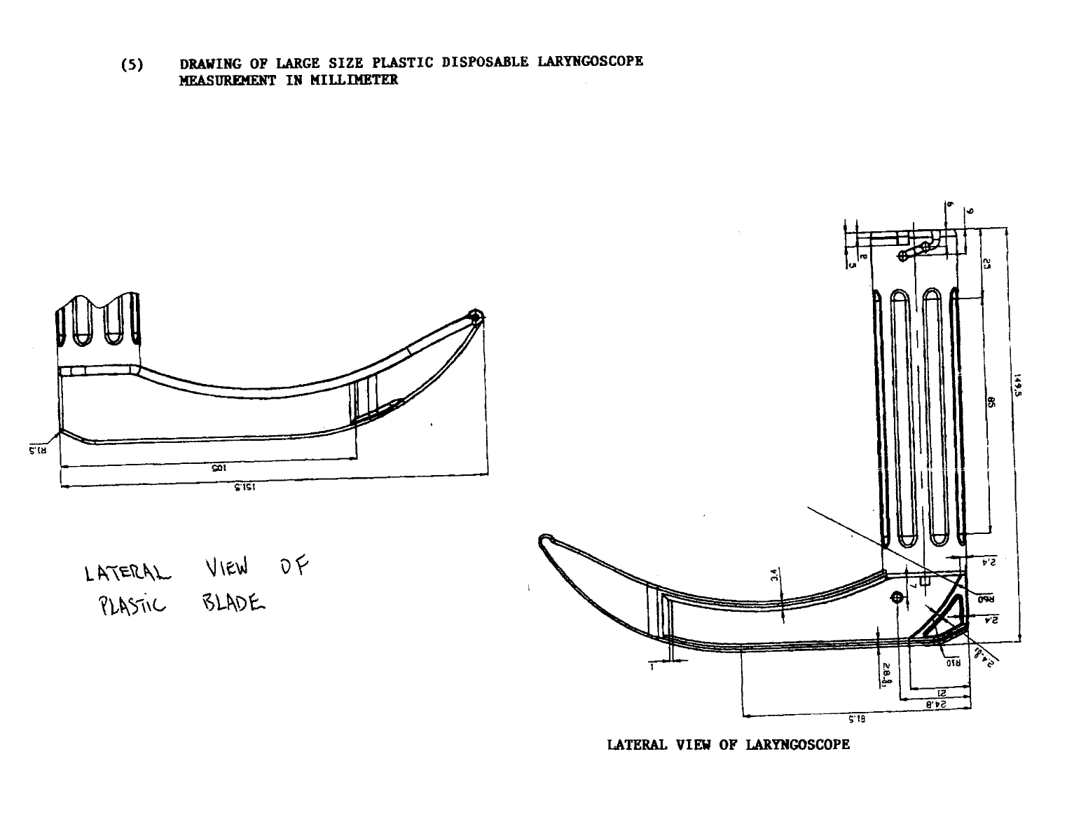

This invention relates to the development of a novel disposable plastic laryngoscope with a magnified built in light source for examination of the larynx.

### SUMMARY OF INVENTION

The laryngoscope is an instrument for the examination of the larynx. A laryngoscope is used in the operation room, emergency room, intensive care units and ambulances. Almost all laryngoscopes are metal with metal blades. The downside to this is that after each use it needs to be sterilized and repackaged. These measures are costly. Therefore, a novel disposable plastic laryngoscope with built in light sources was invented. This invention (disposable plastic laryngoscope) is easy to operate and has an affordable price with magnified light sources that allow physicians and other operators to have a clear view of the larynx.

### DETAILED/DESCRIPTION OF THE INVENTION

This invention relates to the development of the novel plastic disposable laryngoscope powered by two ‘C’ batteries or any other power source—internal or external (e.g. electro magnetic power). This invention consists of three componenets. The first component—plastic rotational light switch cover (which can also change to a press button or any other form) contains a metal spring and a metal connector. The second component—plastic handle in different shapes and sizes that contain two “C” batteries or any other power source variation—internal or external as well as metal connector and a light bulb or any other light source. The third component is a transparent plastic blade of different lengths and shape, which is connected to the base of the plastic handle. A plastic or glass refractory mirror is positioned in the plastic blade to refract the light sources along the plastic blade and to the end of the blade. The light intensity delivered by this novel disposable plastic laryngoscope is double the candlelight power when compared to other commercially available laryngoscopes.

Drawing one and drawing two with further show the above discussion as it relates to the invention. The laryngoscope comes in different sizes: small, medium and large depends on the length of the plastic transparent blade. Drawing three depicts the small size. Drawing four depicts the medium size and drawing five depicts the large size.

This invention is further illustrated by referring to the accompanying drawings of which—

Drawing three depicts the small size plastic sized disposable laryngoscope with measurements.

Drawing four depicts the medium plastic sized disposable laryngoscope with measurements.

Drawing five depicts the large plastic sized disposable laryngoscope with measurements.

What is claimed:

1. A novel disposable plastic laryngoscope

2. A novel disposable plastic laryngoscope with built in power source and built in refractory light sources. This power source can consist of any internal or external means of energy.

3. A novel disposable plastic laryngoscope with magnified light sources; e.g. double or more candle light intensity when compared to regular metal or any other laryngoscope.

4. A novel disposable plastic laryngoscope which contains a plastic transparent blade that varies in length and width.

5. A novel disposable plastic laryngoscope containing plastic or glass refractory mirror at different or specific angles (e.g. 45 degrees) to refract the light sources and magnify intensity.
